(19) United States
(12) Patent Application Publication
(10) Pub. No.: US 2019/0142598 A1
(43) Pub. Date: May 16, 2019

(54) ULTRATOE, VERSION III, ORTHOPEDIC TOE IMPLANT SYSTEM

(71) Applicant: RICHARD DONALD KOENIG, BOYNTON BEACH, FL (US)

(72) Inventor: RICHARD DONALD KOENIG, BOYNTON BEACH, FL (US)

(73) Assignee: RICHARD DONALD KOENIG, BOYNTON BEACH, FL (US)

(21) Appl. No.: 16/034,842

(22) Filed: Jul. 13, 2018

(51) Int. Cl. A61F 2/42; A61B 17/15; A61B 17/16; A61B 17/17

(52) U.S. Cl. CPC A61F 2/4225; A61B 17/15; A61L 27/045; A61B 17/1775; A61F 2002/4233; A61B 17/1682

(57) ABSTRACT

A second generation, version III prosthetic joint for replacing a metatarsophalangeal joint of a human foot has a metatarsal head component with a convex bearing surface that rotates, inverts and everts, and slightly abducts and adducts; normal movement of a healthy human great toe joint, against the concave surface of the phalangeal base second component. The base is elliptical and formed by two different radii of curvature with a desired ratio and constructed of high density medical grade polyethylene. The internal surface is titanium alloy with an intentionally irregular roughened surface for increased bone attraction and increased surface tension. The stem is a shortened square alloy peg. also roughened. The metatarsal head component has an internal surface for confronting the bone with a four chamfer flat surface configuration, one surface is horizontal and parallel to the long axis of the metatarsal bone, another extends approximately perpendicular thereto, and two others extend at angles to the long axis. All of these surfaces are irregularly roughened titanium alloy. Extending from the perpendicular surface, are two parallel short cylindrical pegs. These pegs aid in early security to the bone, assist the resistance of angular displacement, and increase the surface contact area with bone. The horizontal lower surface allows better distribution of vertical forces and helps resist separation of the metatarsal implant from the bone. The thinned metatarsal component articular surface has four different regions formed by four different radii of curvature with desired ratios to one another. These four surfaces allow the head and base components to reproduce pivoting of a healthy toe joint. A dedicated method for installing the prosthetic joint with improved accuracy is part of this invention.

There are instruments dedicated to this implant prosthesis that allow for proper alignment and ease of installation even to the relatively inexperienced surgeon providing that the use of said instrumentation has been taught by skilled instructors and proctored to insure understanding of the instrumentation and procedure. The first of such instruments allows for alignment of the guide in the proper plane of the foot. This all-important guide is a first of its kind for this Great foe Joint surgery. From the placement of this guide, all other guides and steps can be accurately placed to insure exactitude and reproducibility. Onto this instrument is added the first of such guides to reduce the subject bone to the dimensions and configuration for implantation. A second guide is then added to the first guide to allow for the preparation of the apposing surface of the subject joint. The two guides are then exchanged for two other corresponding guides that aid in the preparation of the two subject bones for drilling of the holes that will aid in anchoring the implants to bone. Here two specially designed drilling tools are employed to insure that the holes are of proper dimension in all planes and depths. The specially designed drilling tools also are notable for the attention to the prevention of inadvertent guide disruption, which will otherwise contaminate the surgery wound with unwanted metal particles.

### BACKGROUND

### Field of Invention

This invention relates to orthopedic prostheses of the 1st metatarsophalangeal joint of the foot, of the two component, non-constrained concept of implant arthroplasty. The implant is to replace the human great toe joint when that joint has been severely affected by osteoarthritis due to wear and tear, traumatic arthritis, rheumatoid arthritis, gouty arthritis and the arthritis associated with misalignment. This invention serves as the next generation to previous U.S. Pat. No. 5,037,440, issued 6 Aug. 1991.

### BACKGROUND

### Prior Art

Persons who have experienced degenerative joint disease (osteoarthritis) of the 1st metatarsophalangeal joint with pain and loss of motion as well as loss of function of this joint and the entire foot, have historically sought relief through surgery, beginning in the 1st decade of the 20th century. The lexicon of these surgeries includes: resection of a portion of the joint (Keller procedure, 1904), resection of the entire joint (Mayo procedure, 1908) shortening osteotomies to give joint relief, transpositional osteotomies to realign the joint, arthrodesis, fusion to end painful motion (1895), resection of offending reactive bone spurs, various implants to space the joint and of laic (1965), joint replacement implants with some semblance of normal anatomy. This invention is second generation to the orthopedic toe implant U.S. Pat. No. 5,037,440 and has been considered to be the most anatomically correct of its genre. Other implants of similar intention are Reflexion, Bio-Action, and Kinetik Great Toe implant. Historically, as early as 1951, there were surgeon designed implants for the great toe joint: Endler 1951, Swanson 1952, Seeburger 1964, Joplin 1964, Downey 1965. Swanson 1965, 1967, 1974, 1977, 1986, Smith/Weil 1975, Regnauld 1975, Cutter 1971, Johnson 1981, Lawrence 1982, LaPorta 1983, Lubinus 1983, Koenig 1988, Merkle/Sculco 1989, Bio Action 1990. All two component, non-constrained prostheses have as their goal the restoration of non-painful fully functional motion of the foot.

### Objects and Advantages

Accordingly, the objects and advantages of the present invention include:

(a) Anatomically correct 1st metstarsophalangeal joint implant

(b) Two components for the two surfaces of said joint

(c) Thin, articular strikes utilizing modern materials that have demonstrated strength and wear resistance,

(d) bone removal to install said implants,

(e) Two parallel cylindrical pegs to secure the metatarsal implant to the bone and increase surface area for contact with bone,

(f) Convex metatarsal articular surface composed of four radii of curvature with desired ratios to one another to simulate normal human rotational function,

(g) Lower (plantar) convex surface of metatarsal component for better distribution of vertical force and aid to resist displacement of the implant with applied load forces,

(h) Thin phalangeal base implant for minimizing bone removal,

(i) Irregularly roughened titanium alloy phalangeal component back and stem for ease in installation and improved bone interaction (osseo-integration),

(j) Square short stem on phalangeal component for ease in installation,

(k) Stem of phalangeal component and stems of metatarsal component made with simple geometry to allow for machine preparation of their corresponding receiver holes,

(l) Advantage of (k) above to make surgery easier and more reproducible for surgeons, saving time and cost of surgery.

### DRAWING FIGURES

3 views of metatarsal component:

FIG. 1 side configuration with all projections and parallel pegs.

FIG. 2 top view of projections and pegs,

FIG. 3 demonstrating internal surface of component that will be in full contact with bone,

FIG. 4 frontal view as presented in the joint as articular surface,

FIG. 5 top to bottom view demonstrating silhouette of the component

FIG. 6 first step after exposure of the joint, the application of the alignment center is frontal view as presented in the joint as articular surface rod and metatarsal/phalangeal frontal plane cutting guide.

FIG. 7 the application of metatarsal champher cutting guide, replacing the frontal plane guide from above. This guide is positioned over the pin employed in frontal plane guide.

FIG. 8 prepared 1st metatarsal for the next step.

FIG. 9 metatarsal hone with drilling guide in place, once again positioning of prior installed pin.

FIG. 10 drilled and prepared metatarsal for implantation step to follow.

FIG. 11 phalanx is prepared to accept phalangeal component, with use of phalangeal positioning and reaming guide.

FIG. 12 both metatarsal and phalangeal component installed.

FIG. 13 the installation of both metatarsal and phalangeal components

FIG. 14 s the installation of both metatarsal and phalangeal components

FIG. 15 top view of metatarsal component installation

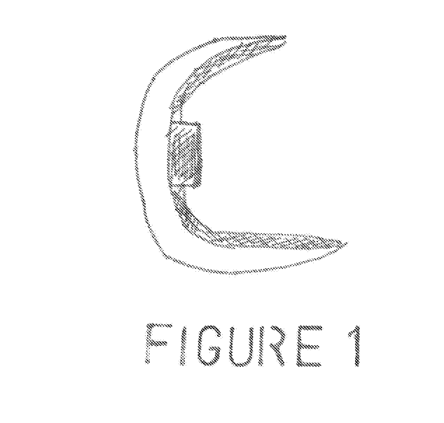

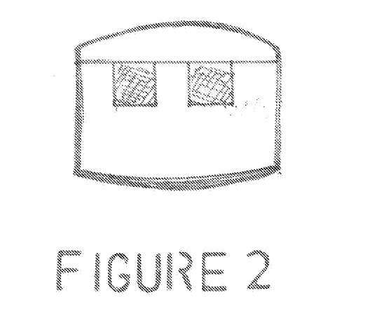

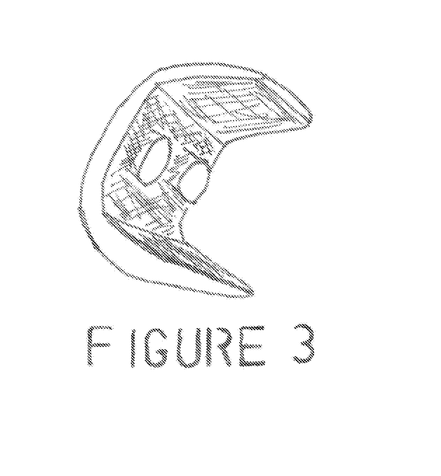

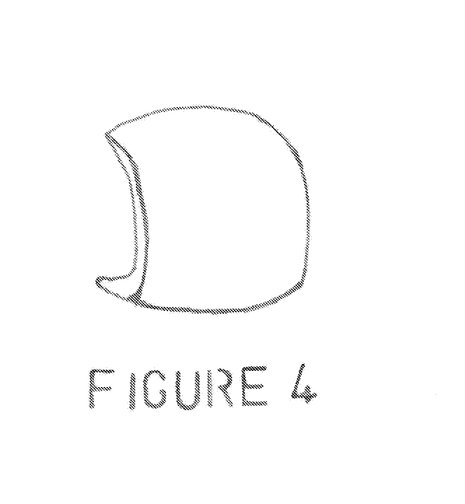

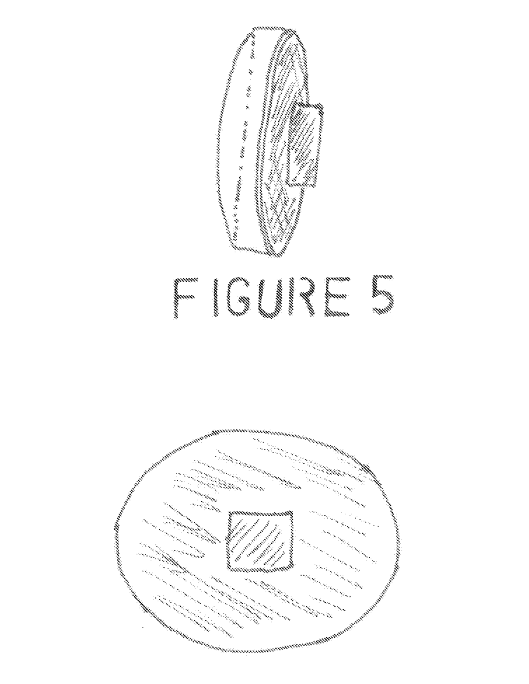

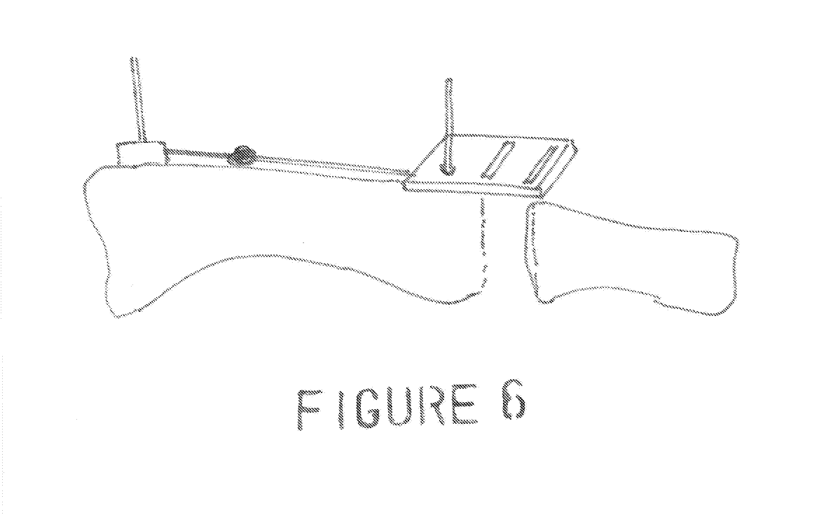

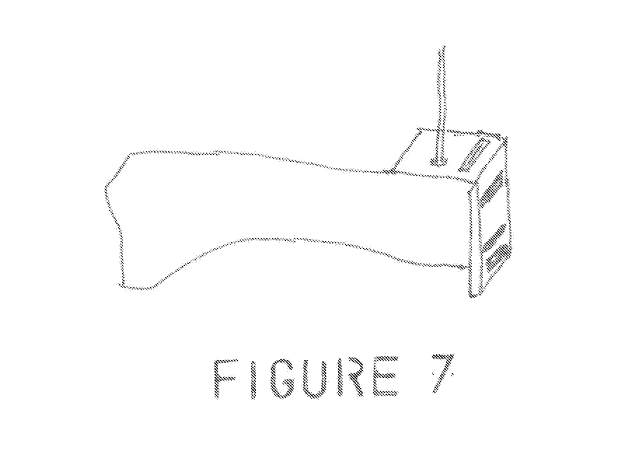

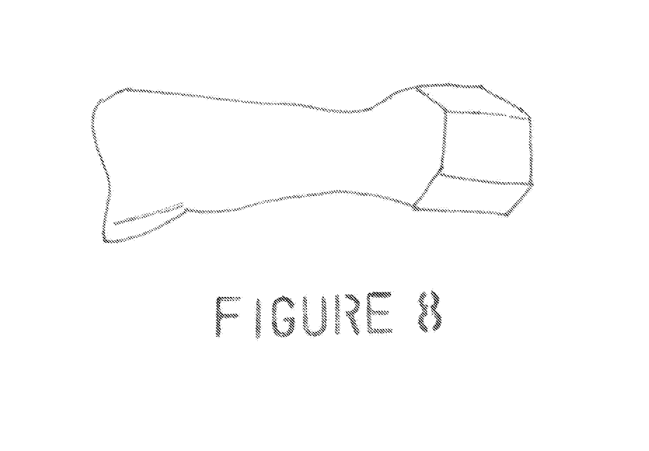

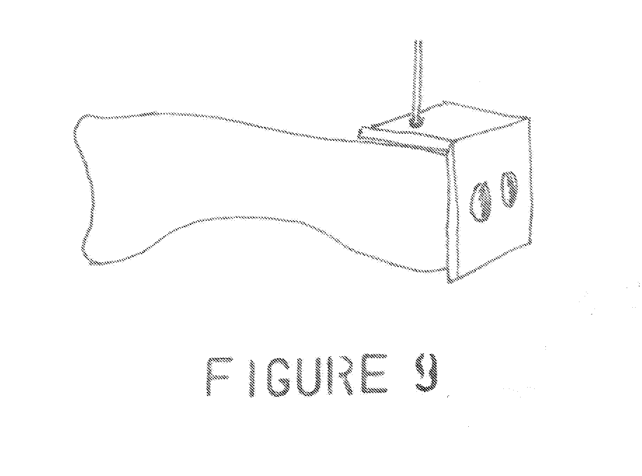

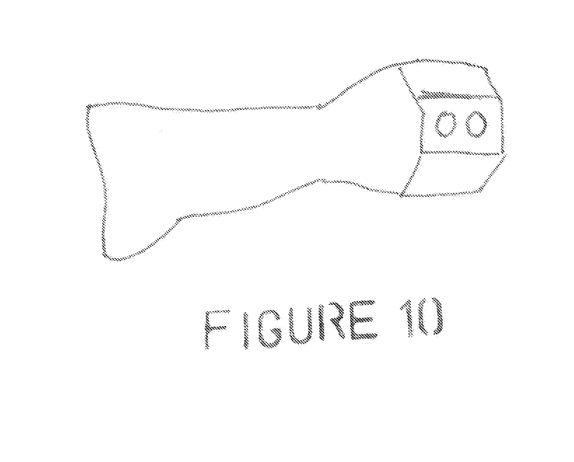

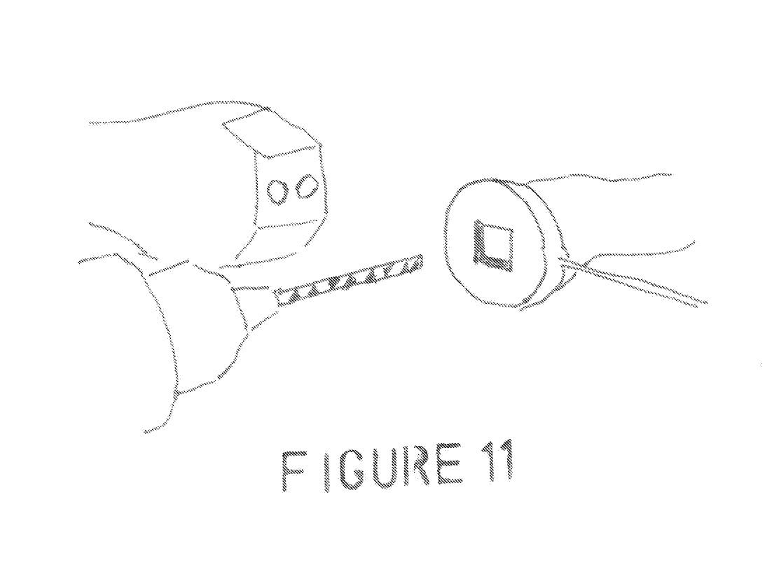

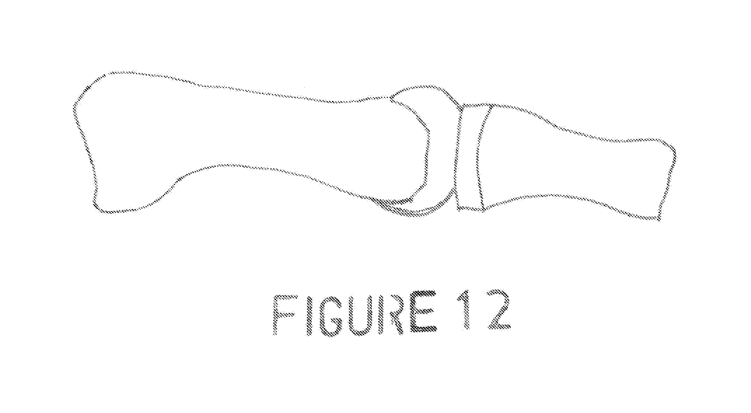

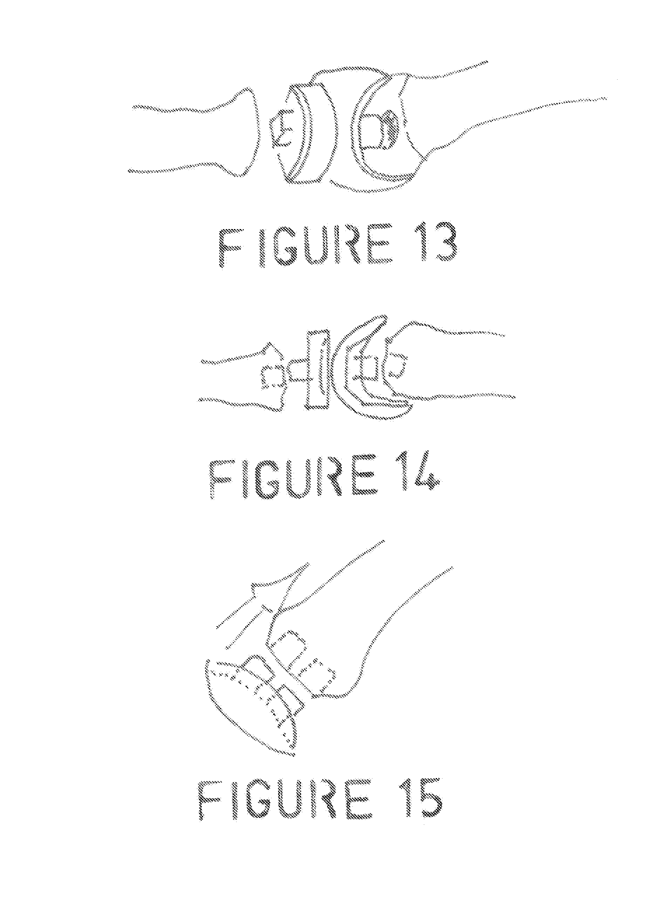

### Operation

UltraToe joint replacement is accomplished with the aid of dedicated instruments when properly used, create precision cuts into bone for the removal of diseased joint surfaces while preparing the surfaces for implantation.

The joint is opened in traditional manner on the medial surface of the foot. The joint and its component bones are fully exposed by underscore of the surrounding ligament. A specially designed instrument is employed to align the 1st metatarsal and its anatomical head. The proximal portion of this guide is positioned over the dorsal prominence (tuberosity) at the base of the first metatarsal and pinned in place. The adjustable arm follows the long axis of the metatarsal. A guide is affixed to the dorsum of the metatarsal alignment instrument, positioned for the desired length, and held in place with surgical stainless steel pins installed through pre-determined holes in the guide and introduced into bone. Through slots, a power sagittal saw is introduced and appropriate sections of bone and diseased cartilage are removed. An extension of this guide is employed to resect a measured amount of the base from the proximal phalanx. The first guide is then removed. A second guide replaces the first guide. It Is used for preparing the metatarsal configuration to match the internal surfaces of the designated implant. The second slotted guide enters the frontal plane of the joint.

A third guide, replaces the second and has two parallel holes in its frontal plane instead of cutting slots, for precision drilling. A collared cylindrical drill especially designed for this surgery with cutting edges only on the front of the cylinder is introduced into the holes in the guide and two precision holes are drilled into the bone stopped at a precise depth. The guide and pins are removed. The resultant holes in bone are 10 percent smaller than the corresponding pegs on the implant but are the same depth dimension.

A fourth guide is applied to the remainder of the end of the proximal phalanx. This guide has a square hole in its center to accommodate a side cutting drill and collar of measured depth. The drill follows the edge of the hole and then is used to ablate the center bone in the hole.

Once again, the guide is removed and the created hole in bone is 10 percent smaller than the stem of the intended implant. The implants are installed with driver and mallet, the joint is rearticulated and closed.

### Summary, Ramifications, and Scope

Thus the reader will see that said UltraToe great toe joint implant and instrumentation has great value to the treating foot and ankle surgeon and the patient being treated. Said invention is reliable, effective, reproducible and assists die competent surgeon to be efficient. This keeps cost of surgery down, the possibility of infection reduced, and the chance for a successful and rapid recovery increased.

Said invention allows for the return of function of the great toe joint when the surgery outcome is optimal. That is, the joint is capable of the three planes of motion traditionally assigned to this joint dorsi/plantar flexion in the sagittal cardinal plane of the human body, adduction/abduction in the transverse cardinal plane of the human body, and inversion/eversion rotation in the frontal cardinal plane of the human body. Having these three ranges of motion resurrected returns function and thus comfort to the recipient.

To a lesser degree, but not less important to the patient, one can choose and wear a greater variety of shoe gear and heel height. Said invention is capable of improving the quality of life for the person in need of this form of medical care.

We claim that the UltraToe System III joint replacement is: a practical orthopedic small joint prosthesis:

1. Comprised of two units, wherein said first unit comprises a metatarsal head articular surface:
a) made of cobalt/chrome alloy and Titanium
b) a means of installing and maintaining the devise into the metatarsal bone made of cobalt/chrome with titanium osseo-integration surfaces,
c) the shape of said devise is “C” shaped on its side view consisting of multiple radii of curvature with internal champhered surfaces for increased surface area and
d) two parallel short cylinders emanating from the frontal plane champher.
e) unique to this design is the lower (plantar) flange for absorbing weight bearing force as well as establishing an abutment plate to resist upward migration of the prosthesis through the bone.

2. Said second unit constitutes a phalangeal base articular surface:
a) made of Ultra High Molecular Weight Polyethylene
b) married to a base and stem made of titanium far installing and maintaining the devise into the shaft of ,the proximal phalanx bone.

3. Said invention has dedicated instrumentation for the purposes of bone preparation to receive said invention and aid in the installation of said invention components into patient bone:
a) a metatarsal alignment that precisely sets up the stage for surgery of the First Metatarsophalangeal joint
b) a metatarsal preparation cutting guide with capture slots for power blade insertion,
c) a metatarsal preparation guide with precision parallel drilling holes and a corresponding drill bit with depth restricting collar,
d) a phalanx base cutting guide with multipurpose to aid in the resection of the phalanx base and develop the hole in the center of the remaining bone to accept the step of the devise phalanx component.
